(19) United States
(12) Patent Application Publication
(10) Pub. No.: US 2007/0134721 A1
(43) Pub. Date: Jun. 14, 2007

(54) Sensor

(76) Inventors: Peter G. Laitenberger, Cambridge (GB); Stuart P. Hendry, Cambridgeshire (GB); Gavin L. Troughton, Cambridge (GB)

Correspondence Address: BROMBERG & SUNSTEIN LLP, 125 SUMMER STREET, BOSTON, MA, 02110-1618, US

(21) Appl. No.: 10/588,172

(22) PCT Filed: Feb. 3, 2005
(86) PCT No.: PCT/GB05/00367

§ 371 Date: Aug. 1, 2006

(30) Foreign Application Priority Data
Feb. 3, 2004 (GB) 0402326.3
Feb. 3, 2004 (GB) 0402324.8
Mar. 3, 2004 (GB) 0402325.5
Feb. 3, 2004 (GB) 0402327.1
Feb. 3, 2004 (GB) 0402323.0
Mar. 4, 2004 (GB) 0404924.3
Mar. 9, 2004 (GB) 0405313.8
Mar. 9, 2004 (GB) 0405312.0
Apr. 16, 2004 (GB) 0408535.3

(51) Int. Cl. G01N 33/53; C12M 3/00

(52) U.S. Cl. 435/7.1; 435/287.2; 977/902

(57) ABSTRACT

This invention relates to a sensor suitable for detecting the presence of one or more analytes. The sensor comprises a substrate; a confinement structure disposed on the substrate, wherein the confinement structure comprises at least a first limiting structure defining a first interior space; a transducer proximal to the first interior space; and a first synthetic polymer capable of selectively binding a first analyte, within the confinement structure. The confinement structure may have a second or further limiting structure defining a second or further interior space containing the first or preceding interior space. The sensor may also have additional confinement structures containing different materials for detecting additional analytes or taking reference measurements.

This invention relates to a sensor and in particular to a sensor for the detection of biologically important species.

Modern healthcare relies extensively on a range of chemical and biochemical analytical tests on tissue samples and a variety of body fluids to enable early recognition, diagnosis and management of disease. Accordingly, there is a significant market for in vitro diagnostics, including sensors.

Medical and technological advances have considerably expanded the scope of diagnostic testing and an increasing understanding of the molecular interactions in the human body, together with the emergence of developing technologies, such as Microsystems and nanotechnology, is revolutionising diagnostic technology. There is also a proliferation of point-of-care (PoC) testing which is relevant in situations where a rapid response is a prime consideration allowing therapeutic decisions to be made quickly.

Despite recent advances in PoC testing, several compelling needs remain unmet. These include the requirement for an increased speed and frequency of test procedures, the simultaneous monitoring of an ever-growing range of analytes, and the ease of sample preparation and of result interpretation.

Many of the presently available diagnostic tests rely almost exclusively on the use of sophisticated biological receptors, such as enzymes, antibodies and DNA, as the recognition element in the test. Due to their biological derivation, these biomolecules typically suffer from a number of limitations when used in sensing applications. These limitations include instability, which is particularly apparent during manufacture and sterilisation; the high price of biological receptors; their poor performance in non-aqueous media; their sensitivity to environmental factors, such as pH, ionic strength, temperature etc.; the limited number of natural biological receptors for important analytes; their variability (due to their method of derivation); and their poor compatibility with micromachining (or microfabrication) and miniaturisation technologies.

A promising route to overcome these issues is offered by synthetic polymer-based receptors, such as molecularly imprinted polymers (MIPs). Synthetic receptors avoid many of the disadvantages associated with biological receptors. Molecular imprinting, for example, is a generic and cost-effective technique for preparing synthetic receptors which combine high affinity and high specificity with robustness and low manufacturing costs. In addition, MIP receptor materials have already been demonstrated for a wide range of clinically relevant compounds and diagnostic markers. In contrast to biological receptors, synthetic receptors, and particularly MIPs, are typically stable at low and high pH, pressure and temperature; are inexpensive and easy to prepare, tolerate organic solvents; are relatively straightforward to design and may be prepared for practically any analyte; and are fully compatible with micromachining (or microfabrication) and miniaturisation technology. Three particular features have made MIPs the target of intense investigation, namely their high affinity and selectivity, which are similar to those of natural receptors, their unique stability, which is superior to that of natural biomolecules, the simplicity of their preparation and the ease of adaptation to different practical applications.

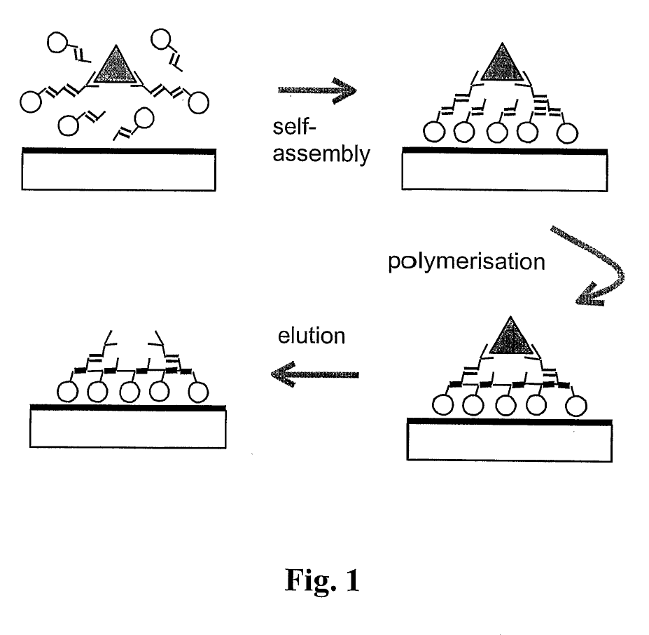

Molecular imprinting may be defined as the process of template-induced formation of specific recognition sites in a material, where the template directs the positioning and orientation of the material's structural components by self-assembly mechanisms. FIG. 1 shows a schematic representation of the self-assembly of a MIP from monomeric starting materials to form a polymer having binding sites with specificity for the template, shown as a triangle, and the subsequent elution of the template. A wide range of chemical compounds have been imprinted successfully using this technique, ranging from small molecules (up to 1200 Da), such as small organic molecules (e.g. glucose) and drugs, to large proteins and cells.

A number of methodologies have been proposed to introduce synthetic polymer-based receptors, including MIPs, into devices for the analysis of clinically relevant analytes but to date have only had limited success.

Petcu M et al in Molecular imprinting of a small substituted phenol of biological importance, Analytica Chimica Acta 435 (2001) 49-55 and Propofol-imprinted membranes with potential applications in biosensors, Analytica Chemica Acta 504 (2004) 73-79 discloses a system for the detection of the intravenous anaesthetic propofol. These documents disclose the synthesis of a MIP capable of binding propofol. The former document discusses the absorption of propofol in a methanol solution on to the MIP. The latter document discloses the attachment of the MIP to a number of different supports, including nylon, cellulose ester, glass and PTFE, and the subsequent absorption of propofol in a methanol solution on to the MIP. In both cases, the concentrations of propofol in the samples before and after exposure to the MIP are determined using HPLC. However, such methods tend to suffer from a number of disadvantages, including being off-line, tending to be cumbersome to carry out, requiring the use of additional chemicals and being generally slow to use.

GB 2 337 332 discloses an electrode having a surface modified with a synthetic polymer which has been functionalised to enable the polymer to bind specifically to an analyte. This document discloses MIPs and their use for the detection of phenolic analytes. The synthetic polymer may be attached to the electrode by a number of techniques, including dip coating, spray coating, spin coating, screen printing and jet printing. The sensor is then contacted with the analyte being detected and the analyte is detected selectively by electrochemical means. A sensor such as that disclosed in this document suffers from a number of potential drawbacks, for example, the peeling of the MIP from the surface of the substrate. Moreover, if a sensor is used for the direct analysis of biological fluids, such as urine, blood, interstitial fluids, cerebral fluids and dialyte, a wide range of cells, platelets, proteins and other substances will tend to bind to the surface of the MIP causing fouling of the sensor which reduces the active area thereby reducing the sensitivity of the sensor. This is a particular problem if the sensor is intended for continuous or semi-continuous use or for multiple uses.

There remains, therefore, a requirement in the art for a sensor capable of functioning in a clinical setting.

Accordingly, the present invention provides a sensor comprising a substrate; a confinement structure disposed on the substrate, wherein the confinement structure comprises at least a first limiting structure defining a first interior space; a transducer proximal to the first interior space; and a first synthetic polymer capable of selectively binding a first analyte, within the confinement structure.

The present invention also provides a method of detecting a target species in a sample comprising contacting a sensor as defined hereinabove with a sample containing or suspected to contain the target species.

The present invention will now be described with reference to the accompanying drawings, in which:

FIG. 1 shows a schematic representation of the self-assembly process;

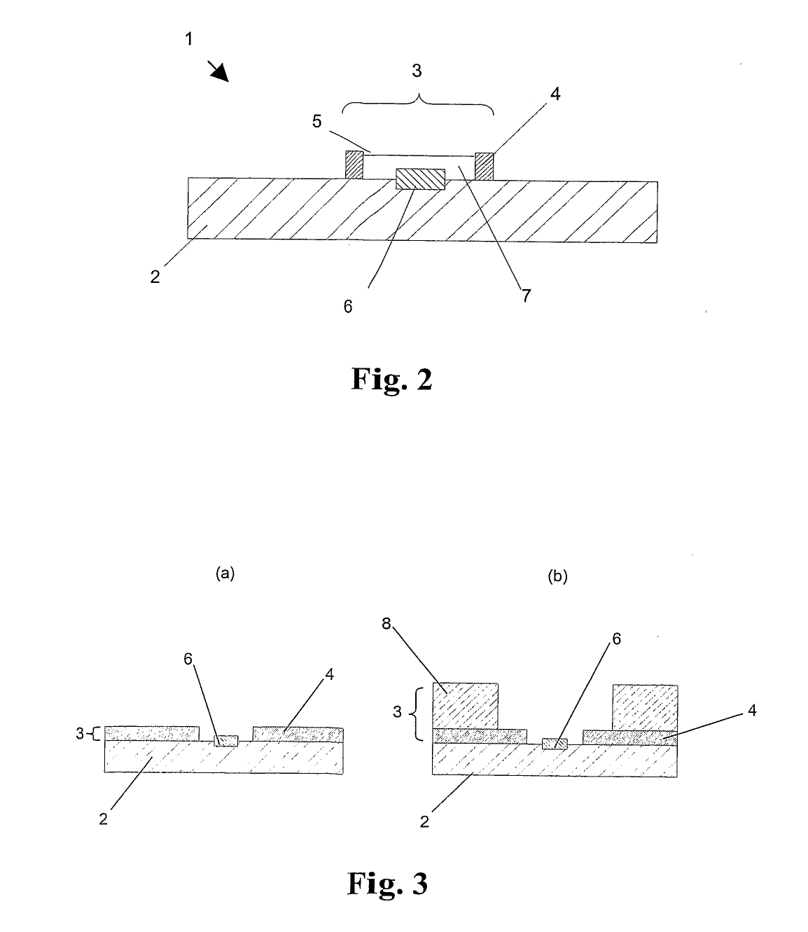

FIG. 2 shows a sensor in accordance with the present invention;

FIG. 3 shows a sensor in accordance with the present invention having (a) a single limiting structure and (b) a double limiting structure;

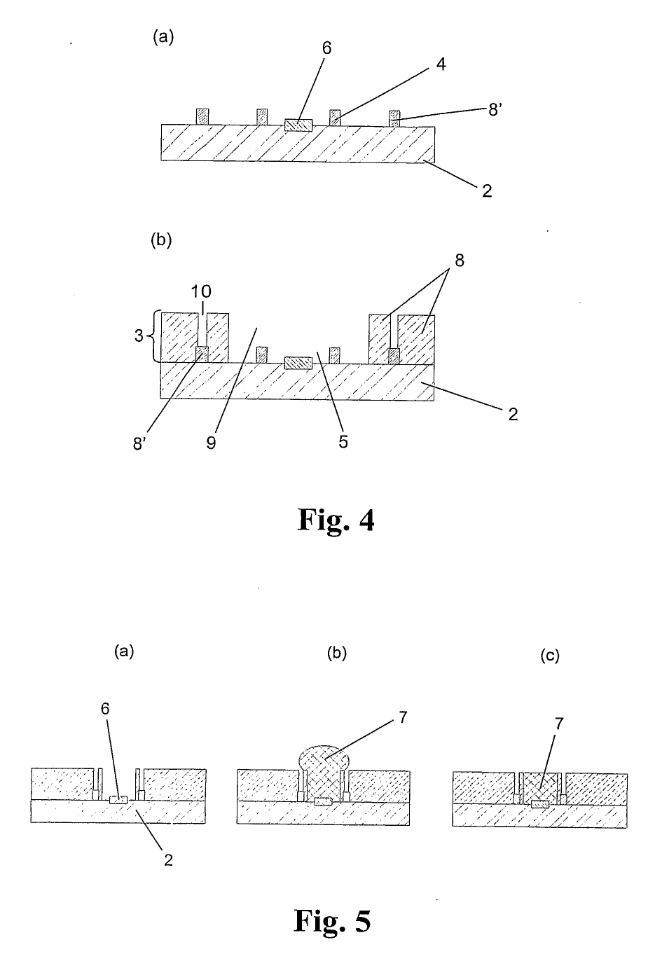

FIG. 4 shows stages of the fabrication of a sensor in accordance with the present invention;

FIG. 5 shows stages in the addition of a material into the confinement structure in accordance with the present invention;

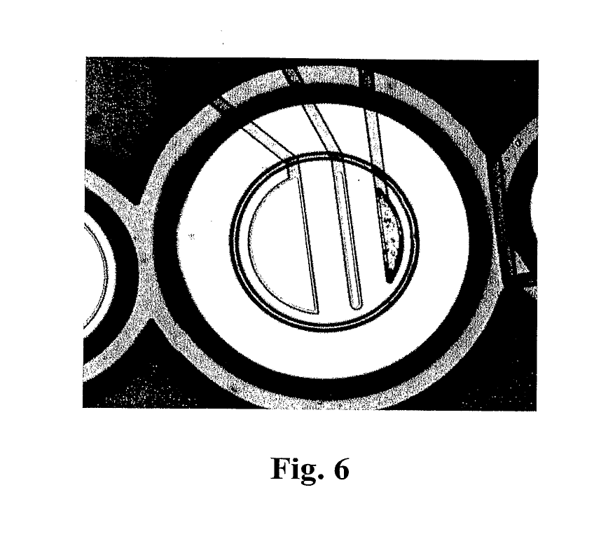

FIG. 6 shows a photograph of a sensor of the present invention incorporating an amperometric transducer;

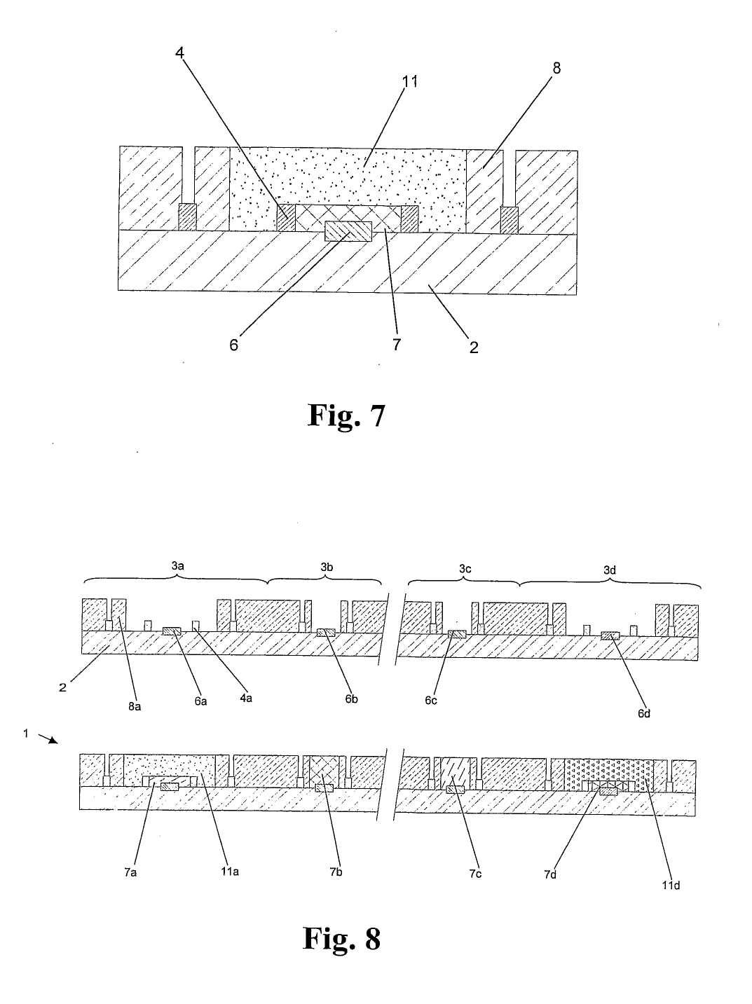

FIG. 7 shows a sensor in accordance with the present invention having a pot and lid structure;

FIG. 8 shows a sensor in accordance with the present invention having a plurality of confinement structures;

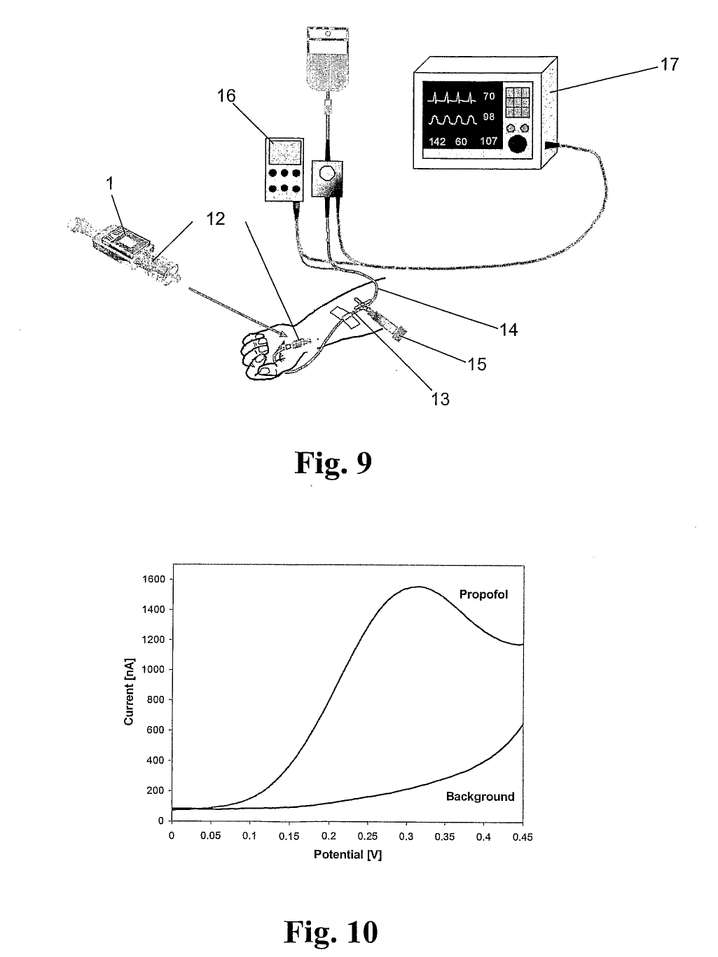

FIG. 9 sensor in accordance with the present invention incorporated into a intravenous monitoring system; and

FIG. 10 shows a voltammetric scan of a 6 μM propofol solution obtained using sensor in accordance with the present invention.

As shown in FIG. 2, the sensor 1 of the present invention includes a substrate 2 and a confinement structure 3 disposed on the substrate. The confinement structure has a first limiting structure 4 defining a first interior space 5. This may be likened conceptually to a “pot”. A transducer 6 is also disposed proximal to the first interior space 5. A first synthetic polymer capable of selectively binding a first analyte, e.g. a molecularly imprinted polymer (MIP), 7 is also included within the confinement structure 3, preferably within the first interior space 5.

The synthetic polymer may be any synthetic polymer provided the polymer is capable of selectively binding an analyte (i.e. it functions as a receptor). The selective binding may be a result of functional groups on the polymer which interact with a specific analyte. The synthetic polymer preferably comprises one or more functionalised monomers and one or more cross-linkers. A preferred polymer is a molecularly imprinted polymer (MIP).

Molecularly imprinted polymers (MIPs) are essentially artificial macromolecular receptors prepared by molecular imprinting of synthetic polymers. MIPs are prepared by polymerising functional monomers or copolymerising functional and cross-linking monomers in the presence of an imprint molecule which acts as a molecular template. The functional monomers initially form a complex with the imprint molecule and, following polymerisation, their functional groups are held in position by the highly cross-linked polymeric structure. In this way, a molecular memory is introduced into the polymer which is then capable of binding the imprint molecule. The imprinting of small organic molecules is now well established in the art.

The functional monomer should be capable of binding to the imprint molecule, via functional groups on the functional monomer. Binding may be via a covalent bond or by intramolecular forces, such as a hydrogen bond or van der Waals forces. A suitable functional group may be, for example, a carboxylic acid in a (meth)acrylic acid ester, although the nature of the functional group will depend on the nature of the imprint molecule. The monomer must, of course, be polymerisable and able to react with a cross-linker when present. Suitable monomers include, but are not limited to, acrylic monomers, such as (meth)acrylic acid, (meth)acrylic acid esters, (meth)acrylamide, (meth)acylonitrile, 2-hydroxyethylmethacrylate (HEMA), N,N,N-triethylaminoethyl(meth)acrylate, trifluoromethylacrylic acid, acrylamide, n,n-methylenebisacrylamide, acrylonitrile, 2-acrylamido-2-methyl-1-propanesulfonic acid acrolein, ethylene glycol dimethacrylate, imidazole-4-acrylic acid ethyl ester, imidazole-4-acrylic acid, 2-(diethylamino)ethyl methacrylate; vinyl and allyl monomers, such as 2- and 4-vinylpyridine, m-and p-divinylbenzene, styrene, aminostyrene 1-vinylimidazole, allylamine; urethanes; phenols; boronates, such as aminophenyl boronate; amines, such as phenylene diamine, phenylene diamine-co-aniline; organosiloxane monomers; carbonate esters, such as methylenesuccinic acid; sulfonic acid; or mixtures (i.e. co-polymers) thereof. See M. Komiyama et al. Molecular Imprinting: From Fundamentals to Applications, Wiley-VCH Verlag GmbH & Co KGBA, Weinleim (2003), G. Wulff Angew. Chem. Int. Ed. Engl. 34, 1812 (1995), and S. Subralumanyam et al. Biosensors & Bioelectronics 16, 631 (2001).

The cross-linker may be included to fix the template-binding sites firmly in the desired structure as well as to influence the porosity of the MIP. The cross-linker must be capable of reacting with the functional monomers to cross link the polymer chains and the cross-linker should preferably be of similar reactivity to the monomer. Suitable cross-linkers include, but are not limited to, ethylene glycol dimethacrylate (EDMA), glycerol dimethacrylate (GDMA), trimethylacrylate (TRIM), divinylbenzene (DVB) (which is particularly suitable for cross linking acrylate- and vinyl-containing functional monomers), methylenebisacrylamide and piperazinebisacrylaniide (which are particularly suitable for cross linking acylamides), phenylene diamine (which is particularly suitable for cross linking amines such as aniline and aminophenyl boronate), dibromobutane, epichlorohydrine, trimethylolpropane trimethacrylate and N,N′-methylenebisacrylamide.

The mole ratio of functional monomer to cross-linker is preferably from 1:1 to 1:15. Mixtures of monomers and cross-linkers may also be used.

The functional monomer and/or the cross-linker may act as a solvent for the polymerisation reaction or an additional solvent may be added. Suitable solvents are known in the art and include DMSO (dimethyl sulfoxide), formic acid, acetic acid, DMF (dimethylformamide), methanol, acetonitrile, dichloromethane, chloro>form, THF (tetrahydrofuran), toluene and cyclohexane. Mixtures of these solvents may also be used to obtain the desired solvation and porogenic properties.

The polymer preferably has a molecular weight from 1 to 100,000 kDa, more preferably 10 to 10,000 kDa and most preferably 10 to 5,000 kDa.

A range of MIPs has been developed for clinically relevant assays and sensors. The following is a non-exhaustive list of such MIPs: amino acids and derivatives (Panasyuk T L et al. Electropolymerized molecularly imprinted polymers as receptor layers in capacitive chemical sensors. Anal Chem 1999; 71: 4609-4613; Liao Y, et al. Building fluorescent sensors by template polymerization: the preparation of a fluorescent sensor for L-tryptophan Bioorg Chem 1999; 27: 463-476; and Peng H et al. Development of a thickness shear mode acoustic sensor based on an electrosynthesized molecularly imprinted polymer using an underivatized amino acid as the template. Analyst 2001; 126: 189-194); aniline and phenol derivatives (Piletsky S A et al. Chemical grafting of molecularly imprinted homopolymers to the surface of microplates. Application of artificial adrenergic receptor in enzyme-linked assay for β-agonists determination. Anal Chem 2000; 72: 4381-4385 and Morita M et al. Integrated array microelectrodes as electrochemical sensors Electrochim Acta 1997; 42: 3177-3183); anions and cations (Murray G M et al. Molecularly imprinted polymers for the selective sequestering and sensing of ions J Hopkins Apl Tech Dev 1997; 18: 464-472 and Hutchins R S et al. Nitrate-selective electrode developed by electrochemically mediated imprinting doping of polypyrrole Anal Chem 1995; 67: 1654-1660); barbituric acid (Mirsky V M et al. Spreader-bar approach to molecular architecture: formation of artificial chemoreceptors Angew Chemie bitem Ed 1999; 38/8: 1108-1110); caffeine (Kobayashi T et al. Molecular imprinting of caffeine and its recognition assay by quartz-crystal microbalance Anal Chim Acta 2001; 435: 141-149 and Lai E P C et al. Surface plasmon resonance sensors using molecularly imprinted polymers for sorbent assay of theophylline, caffeine, and xanthine Can J Chem 1998; 76: 265-273); chloramphenicol (Levi R et al. Optical detection of chloramphenicol using molecularly imprinted polymers Anal Chem 1997; 69: 2017-2021); cholesterol (Piletsky S A et al. Molecularly imprinted self-assembled films with specificity to cholesterol Sensor Actuat B 1999; 60: 216-220); cinchola alkaloids (Matsui J et al. Molecularly imprinted fluorescent-shift receptors prepared with 2-(trifluoromethyl) acrylic acid Anal Chem 2000; 72: 3286-3290); clenbuterol (Pizzariello A et al. A solid binding matrix/molecularly imprinted polymer-based sensor system for the determination of clenbuterol in bovine liver using differential-pulse voltammetry. Sensor Actuat B-Chem 2001; 76: 286-294); epinephrine (Piletsky S A et al. Chemical grafting of molecularly imprinted homopolymers to the surface of microplates Application of artificial adrenergic receptor in enzyme-linked assay for β-agonists determination Anal Chem 2000; 72: 4381-4385); β-estradiol (Rachkov A et al. Fluorescence detection of beta-estradiol using a molecularly imprinted polymer. Anal Chim Acta 2000; 405: 23-29); flavonol (Suarez-Rodriguez J L et al. Flavonol fluorescent flow-through sensing based on a molecular imprinted polymer Anal Chim Acta 2000; 405: 67-76); gases (Kodakari N et al. Molecular sieving gas sensor prepared by chemical vapour deposition of silica on tin oxide using an organic template Bul Chem Soc Jpn 1998; 71: 513-519); morphine (Ansell, R J et al. Molecularly imprinted polymers for bioanalysis: chromatography, binding assays and biomimetic sensors Current Opinion in Biotechnology 7 (1996) 89-94, Anderson, L I et al. Mimics of the binding sites of opioid receptors obtained by molecular imprinting of enkephalin and morphine Proceedings of the National Academy of Sciences 92 (1995) 4788-4792, Kröger, S et al. Affinity electrode for electrochemical analysis GB 2337 332, Kriz D et al. Competitive amperometric morphine sensor based on an agarose immobilised molecularly imprinted polymer. Anal Chim Acta 1995; 300: 71-75); nicotine (Tan Y et al. A study of a new TSM bio-mimetic sensor using a molecularly imprinted polymer coating and its application for the determination of nicotine in human serum and urine Bioelectrochemistry 2001; 53: 141-148); nucleic acids and derivatives (Turkewitsch P et al. Fluorescent functional recognition sites through molecular imprinting. A polymer-based fluorescent chemosensor for aqueous cAMP Anal Chem 1998; 70: 2025-2030 and Spurlock L D et al. Selectivity and sensitivity of ultrathin purine-templated overoxidized polypyrrole film electrodes Anal Chira Acta 1996; 336: 37-46); paracetamol (Tan Y G et al. A study of a bio-mimetic recognition material for the BAW sensor by molecular imprinting and its application for the determination of paracetamol in the human serum and urine Talanta 2001; 55: 337-347); phenactin (Tan Y et al. A new system for phenacetin using biomimic bulk acoustic wave sensor with a molecularly imprinted polymer coating. Sensor Actuat B-Chem 2001; 73: 179-184); propranolol (Ye L et al. Polymers recognizing biomolecules based on a combination of molecular imprinting and proximity scintillation: a new sensor concept J Am Chem Soc 2001; 123: 2901-2902); sugars and derivatives (Piletsky S A et al. Imprinted membranes for sensor technology-opposite behavior of covalently and noncovalently imprinted membranes Macromolecules 1998; 31: 2137-2140; Cheng Z et al. Capacitive detection of glucose using molecularly imprinted polymers Biosens Bioelectron 2001; 16: 179-185; Chen G et al. A glucose-sensing polymer Nat Biotechnol 1997; 15: 354-357; and Wang Wet al. Building fluorescent sensors by template polymerization: the preparation of a fluorescent sensor for D-fructose. Org Lett 1999; 1: 1209-1212); terpene (Percival C J et al. Molecular-imprinted, polymer-coated quartz crystal microbalances for the detection of terpenes Anal Chem 2001; 73: 4225-4228); vitamin-K1 (Andersson L I et al. Studies on guest selective molecular recognition on an octadecyl silylated silicon surface using ellipsometry Tetrahedron Lett 1988; 29: 5437-5440); antibiotics: beta-lactam (Skudar, Ket al. Selective Recognition and Separation of beta-Lactam Antibiotics Using Molecularly Imprinted Polymers Anal. Commun., 36 (1999) 327-331), pentamidine (Sellergren, B Imprinted dispersion polymers: A new class of easily accessible affinity stationary phases Journal of Chromatography A, 673 (1994) 133-141 and Direct drug determination by selective sample enrichment on an imprinted polymer Anal. Chem. 66 (1994) 1578-1582, Nilson K, et al. Imprinted polymers as antibody mimetics and new affinity gels for selective separations in capillary electrophoresis Journal of chromatography A 680 (1994), 57-61), chloramphenicol (Levi, R et al. Optical detection of chloramphenicol using molecularly imprinted polymers Anal. Chem. 69 (1997) 2017-2021), erythromycin (Siemann, M et al. Separation and detection of macrolide antibiotics by HPLC using macrolide-imprinted synthetic polymers as stationary phases The Journal of Antibiotics 50 (1997) 89-91), tylosin (Siemami, M et al. Separation and detection of macrolide antibiotics by HPLC using macrolide-imprinted synthetic polymers as stationary phases The Journal of Antibiotics 50 (1997) 89-91), oleandomycin (Siemann, M. et al. Separation and detection of nmacrolide antibiotics by HPLC using macrolide-imprinted synthetic polymers as stationary phases The Journal of Antibiotics 50 (1997) 89-91), vancomycin (Roa, J et al. Using surface plasmon resonance to study the binding of vancomycin and its dimmer to self-assembled monolayers presenting D-Ala-D-Ala J. Amr. Chem. Soc. 121 (1999) 2629-2630, Asanuma, H et al. Tailor-Made Receptors by Molecular Imprinting Advanced Materials 12 (2000) 1019-1030 and Molecular Imprinting of Cyclodextrins Leading to Synthetic Antibodies Journal of Inclusion Phenomena and Macrocyclic Chemistry 44 (2002) 365-367), penicillin G (Cederfur, J et al. Synthesis and screening of a molecularly imprinted polymer library targeted for penicillin G J Comb. Chem. 5 (2003) 67-72), cefazolin (Asanuma, H et al. Tailor-Made Receptors by Molecular Imprinting Advanced Materials 12 (2000) 1019-1030, Asanuma, H et al. Molecular Imprinting of. Cyclodextrins Leading to Synthetic Antibodies Journal of Inclusion Phenomena and Macrocyclic Chemistry 44 (2002) 365-367), and phenethicillin (Asanuma, H et al. Tailor-Made Receptors by Molecular Imprinting Advanced Materials 12 (2000) 1019-1030 and Molecular Imprinting of Cyclodextrins Leading to Synthetic Antibodies Journal of Inclusion Phenomena and Macrocyclic Chemistry 44 (2002) 365-367).

The substrate is preferably a substantially planar substrate although it could be curved. The substrate may be a silicon wafer, ceramic, glass, metal or plastic etc.

Suitable transducers are known in the art and the sensor of the present invention may employ electrochemical (e.g. potentiometric, in particular ion-sensitive field effect transistors (ISFETs) or amperometric), conductimetric, optical, fluorescent, luminescent, absorption, time-of-flight, gravimetric, strain or displacement, surface-acoustic waves, resonant or thermal principles. The transducer 6 is disposed proximal to the first interior space 5. That is, the transducer 6 is sufficiently close to the first interior space 5 that a signal generated in the confinement structure 3 may be received by the transducer 6. Preferably the transducer 6 is disposed on the substrate 3 or within the substrate 3 (e.g. an implanted transducer), more preferably on the substrate 3.

Where the sensor 1 is dependent on the flow of an electrical current through the material sample or polymer, such as the flow of an electrical current between the electrodes of an amperometric or conductinietric transducer, the synthetic polymer 7 must be in electrical communication with the transducer 6, that is, either be sufficiently close to the transducer 6 to allow direct current flow between the analyte and the transducer 6, or the sensor requires the presence of a conducting material. This conducting material may be present in the sample being analysed, e.g. a sample in saline solution, or the conducting material may be included in the first interior space 5. This conducting material may be, for example, a conducting polymer, e.g. PVDF, electrically conducting organic salts, such as N-methylphenazinium cation with tetracyanoquinodimethane radical anion, or an electrolyte. In the case of a conducting polymer; this conducting polymer could also be incorporated into the synthetic polymer capable of binding an analyte. The confinement structure may also include a mediator, i.e. a substance inside the confinement structure, for example in the form of a solution in the innermost structure, which reacts with the analyte to be detected and/or other materials present in the sample, e.g. dissolved oxygen, to form an intermediate species, which then diffuses to the transducer and is detected there. In one preferred embodiment, this reaction with the transducer converts the intermediate species back into the mediator. One particular example for this would be an electrochemical mediator, which transfers electrons for the oxidation or reduction of a target species from the electrode to the target species or vice versa. Examples of such mediators include ferrocenes although many others are known in the art.

There are many suitable compositions for an electrolyte known in the art. A preferred electrolyte is made up from a buffer solution that includes triethylene glycol and a phosphate buffer. An example for this is a solution of 120 μL of a buffer solution (concentration 100 mmol/L, pH 7) and 30 μL of triethylene glycol. It is also preferable to include a planarising additive, such as polyvinylpyrrolidone (PVP). The addition of the planarising agent is particularly advantageous if other materials are to be deposited above this layer. Another suitable electrolyte is a solution of NaCl in an aqueous phosphate buffer. Preferentially, the total ion concentration in the electrolyte should be of the order of 100 mM. Again it is preferred to add PVP to the solution.

The electrolyte solution should be sufficiently dried until it is crystalline or in the form of a gel. Under operating conditions the electrolyte can become moist on exposure to the sample.

FIG. 3 shows a sensor also having one confinement structure 3, although the confinement structure 3 is shown having either (a) a first limiting structure 4 only or (b) a first limiting structure 4 and a second limiting structure 8. The second limiting structure 8 allows an additional cover layer to be included in the confinement structure 3 (see FIG. 7).

The sensors are fabricated using known techniques. See U.S. Pat. No. 5,376,255 and U.S. Pat. No. 5,376,2565 which describe the formation of such structures. The confinement structure may be created by a so-called “backend-process”, i.e. after the transducer has been integrated into or created on the silicon wafer. At that stage, the wafer is covered in a passivation layer, such as a layer made from plasma-enhanced chemical vapour deposition (PECVD)-deposited SiN or other passivation layers, such as oxides, oxynitrides or resist layers. The passivation layer is removed in the area around the transducer in order to provide access for the analytes to be measured.

The first, and optionally further, confinement structures may then fabricated around the transducer(s). These structures may be fabricated from any layer which can be deposited in the right thickness, such as resists, or other materials which can be patterned using microfabrication techniques, for example photo-patterning and etching, and include metal layers, silicon oxide, silicon nitride, resist materials, for example those typically used for microfabrication, including, but not limited to, polyimide (PI) and SU8. Such materials are commercially available. Other techniques, such as embossing, stamping or laser ablation, may also be used. Typically the confinement structures are created by the deposition of a resist material onto the wafer. A preferred resist material is PI, e.g. Durimide 7510.

One particular embodiment of the process will be described with reference to FIG. 4 for a sensor 1 having a single confinement structure 3 which has itself a first 4 and second limiting structure 8 defining a first 5 and second interior space 9 respectively, although the techniques apply equally to any number of limiting structures and confinement structures 3.

In order to create the confinement structure of the double limiting structures 4,8, a layer of PI is spun onto the wafer. After baking, this layer typically has a thickness of about 10 nm to 5 mm, preferably about 1-40 μm, more preferably about 1-10 μm. It is then patterned by photolithography and developed to leave behind the desired features. FIG. 4(a) shows two annular structures including a first limiting structure 4 defining a first interior space 5 and a precursor 8′ of the second limiting structure 8.

A second PI layer is then deposited on the wafer by spin-coating. This layer is usually thicker than the first layer and typically has a thickness of about 10 nm to 5 mm, preferably about 1-500 μm, more preferably 1-100 μm after the baking step. It is again patterned by photolithography and developed to create the desired shape. FIG. 4(b) shows the second limiting structure 8 which has been fabricated on top of the outer of the two annular structures shown in FIG. 4(a). The second limiting structure 8 in FIG. 4(b) includes a recess 10. The recess 10 assists in the application of the MIP as described below and the presence of the precursor 8′ provides a passivation layer across the substrate to prevent any fluid coming into contact with the substrate.

The depth, size, area, volume and shape of the containment structure will depend on the particular application. However, a typical internal diameter of a containment structure having a single limiting structure is about 1-500 μm preferably about 10-350 μm. In the case of a containment structure having a double limiting structure, the typical diameter of the first limiting structure is also 1-350 μm and the typical internal diameter of the second limiting structure is larger at about 5-600 μm, preferably about 50-600 μm. The height of the first and second limiting structure is dependent on the thickness of the first and second layers which are deposited.

Similarly, additional layers may be deposited on the substrate and patterned in order to create additional limiting structures, such as a triple or quadruple limiting structure etc. around the transducer 6. That is, the confinement structure further comprises one or more further limiting structures defining one or more further interior spaces, the one or more further interior spaces each containing the preceding interior space.

The confinement structure and hence the first, second and further limiting structures may be any shape but are preferably annular. Where there are a plurality of limiting structures and they are annular, they form concentric rings.

FIG. 6 shows a photograph of an amperometric transducer surrounded by a confinement structure 3 having a first and second limiting structure 4,8.

The synthetic polymer 7 exemplified by a MIP may be deposited into the containment structure 3 by a manual or automated dispensing routine. Both approaches use a small syringe which deposits one or more small droplets of the desired solution into the respective confinement structure 3. The size of the droplet will depend on the volume of the confinement structure but may be in the region of 0.1-200 nL. While the manual syringe employs the physical displacement of a piston, the automated system uses a pneumatic system to dispense accurately the desired volume. Other examples include ink-jet printing, spotting, dropping etc.

There are three preferred approaches to deposit the synthetic polymer, e.g. MIP, into the confinement structures.

Firstly, a solution containing all of the components required to form the polymer, e.g. selected monomers, template, plasticiser, cross-linker, initiator etc., is prepared. This solution is then dispensed into the respective confinement structure and polymerised. A variety of methods are available to carry out the polymerisation. For example, initiation by electromagnetic radiation (e.g. visible, UV or infrared radiation), chemical initiation (e.g. using 2,2′-azobis(isobutyronitrile), azobis(cyclohexane carbonitrile) or 2,2′-azobis(2,4-dimethylvaleronitirile) as initiators) or initiation by heat (e.g. heating at a temperature of 60-80° C. for 12-48 h). After the polymer is formed the chip surface may be washed to remove the template from the MIP.

In addition, rather than depositing the complete solution in one step, the different components may be deposited serially into the confinement structures prior to polymerisation. Subsequent steps are then the same as the first method.

Secondly, the MIP may be formed by polymerisation prior to deposition. In this case, the MIP is preferably ground up into small particles. Techniques for grinding polymers are known, for example using a centrifugal ball mill. Sieving may be used to achieve the desired particle size. The minimum particle size of the MIP is preferably a diameter of 10 nm, more preferably 100 nm. The maximum particle size of the MIP is preferably a diameter of 40 μm, more preferably 10 μm, most preferably 1 μm. The MIP is then suspended into a liquid and this liquid is dispensed into the confinement structure. Furthermore, the particles of the MIP may be produced in a particular shape, e.g. by moulding, extrusion, forming or stamping, which can then be deposited onto the transducer element. The particles may be primary particles or structures/agglomerates composed of primary particles

Thirdly, the MIP particles may be entrapped in a cross-linked polymer or membrane, e.g. cellulose, PVC, nylon, HEMA and polyHEMA, silicone, siloxane or polyester. In this case, the MIP particles are preferably mixed with a polymer pre-cursor solution and this solution is then be deposited into the containment structure.

In some cases it may be advantageous to dissolve or suspend the material 7 to be deposited in one or more solvents, for example to enable the deposition of particles in the form of a suspension or to adjust the viscosity of the liquid to be deposited. Once deposited, solvents may evaporate. In such cases, it may be desirable to deposit a larger volume in the confinement structures 3 than is ultimately required for the operation of the sensor element. This approach is shown in FIG. 5. FIG. 5(a) shows a sensor 1 having confinement structure 3 in accordance with the present invention. The material 7 is then deposited in the confinement structure. A recess 10 in the first limiting structure 4 prevents the material 7 from spreading any further along the surface of the structure, for example, by surface tension. As shown in FIG. 5(c), after the deposition, if required, the wafer may be dried, for example in an environment with raised temperature and humidity.

Preferably an automated dispenser is used for volume manufacture. The instrument uses a pattern recognition system to locate the individual containment structures on each substrate in order to achieve the required positional accuracy for the dispensing process. It can dispense very low volumes of polymer or cover layer solution or suspension (in the nanolitre range) with high reproducibility (better than 2%). The dispensing instrument is scaleable for volume manufacture and can achieve dispensing speeds of about 200 droplets per min.

In addition to “trapping” the MIP inside the containment structure, the MIP may also be immobilised on the surface of the substrate 2 (inside the containment structure 3) in a number of ways, if required. Immobilisation of adequate functional groups or free radical initiators onto the surface of the sensor may be realised by linking molecules which attach to the surface of the substrate. The covalent attachment of the MIP to the substrate is then performed via coupling reactions between the chemically modified surface and the MIP.

Immobilisation may be achieved on a variety of materials, such as silicon, silicon oxide, silicon nitride and metals, using a wide range of chemistries (see for example Bartlett P N Modification of sensor surfaces, Handbook of chemical and biological surfaces, Edited by Taylor R F and Schultz J S, Institute of Physics Publishing (1996)). Examples of two convenient routes use a silane or thiol. Further polymerisation of the MIP at this level ensures the stable and robust preparation of the sensor.

Silanisation may be performed on any type of surface displaying hydroxyl groups, for example, silicon, silicon oxide and silicon nitride. It involves the covalent bonding of a silane, either a chlorosilane or an alkyloxysilane, to the surface. Silanisation can lead to a termination of the surface by a variety of functional groups, e.g. amines, thiols and carboxyl groups. See, for example, Pavlovic E, Spatially controlled immobilization of biomolecules on silicon surfaces, Acta Universitatis Upsaliensis, Comprehensive Summaries of Uppsala Dissertations from the Faculty of Science and Technology 867, ISBN 91-554-5691-X (2003). These groups can then be reacted with suitably modified functional groups or initiators to anchor the MIP on to the surface, resulting in layers.

Another immobilisation approach involves the creation of an amine-terminated surface, using spontaneous esterification between the silanol groups (Si—O—H) on the surface of the substrate and ethanolamine. The primary amines which are exposed on the surface after this first modification step may then be used for the binding of other functional groups.

In a preferred embodiment of the present invention as shown in FIG. 7, a cover layer 11 is deposited on top of the polymer 7. In one embodiment, this cover layer 11 is a selectively permeable membrane which is permeable to the analyte to be detected or a substance which takes part in a reaction or interaction leading to the detection of the analyte, but prevents the passage of unwanted interferents, e.g. proteins and other chemicals present in the sample, or confers resistance to the deposition of such interferents. The membrane may also allow preferential partitioning of the analyte. This may be likened conceptually to a “lid” on the “pot” structure.

A number of materials may be used to construct the cover layer 11, e.g. silicone, PVC, 2-hydroxyethyl methacrylate (HEMA) resin etc. A Wide range of polymerisable materials may be used as long as they polymerise or harden under conditions which do not damage the receptor or render it ineffective for the required application. The choice of material depends to a large extent on the polymer and the analyte. A preferred membrane is a UV-cross-linked coating. For example a photo-structurisable HEMA resin, e.g. consisting of a solution of 2.374 g of HEMA, 0.025 g of dimethoxyphenylacetophenone (DMAP) and 0.075 g of tetraethylene glycol methacrylate (TEGM) in 60 μl of triethylene glycol.

In another embodiment, this cover layer is the synthetic polymer 7, that is the synthetic polymer is disposed in the second, or one or more further, interior spaces. In this embodiment, the analyte may freely or preferentially dissolve into or diffuses through this layer. The first interior space then includes a conducting material or space for a conducting material to be incorporated when the sensor 1 is in use, for example where the sample itself is a conducting material. This conducting material thereby allows the synthetic polymer 7 to be in electrical communication with the transducer. In use, the analyte binds selectively to the synthetic polymer and a signal is detected by the transducer via electron or ion exchange through the conducting material. A mediator may also be included as described hereinabove.

As well as the first synthetic polymer 7 in the first interior space 5, there may also be included one or more additional components which create a specific environment around the first synthetic polymer 7 in order to improve the performance of the sensor 1. For example, many MIPs have been designed for operation in non-polar, e.g. non-aqueous, media. By providing a local non-polar medium in the confinement structure, these MIPs may be employed in a sensor 1 operating in a polar environment. This is particularly relevant for sensors where the samples are typically aqueous, e.g. urine or blood. A semi-permeable membrane is then formed on top of the confinement structure 3.

This approach may be particularly advantageous for the detection of substances which exist as an emulsion in a polar solvent. One particular example may be certain medical drugs, for example anaesthetics, such as propofol.

In a preferred embodiment, the sensor 1 includes at least one additional confinement structure 3 as defined herein, i.e. comprises a plurality of confinement structures 3. Each confinement structure 3 contains a transducer 6 disposed on the substrate 2 within the first interior space 5 of each of the at least one additional confinement structure 3, as well as a material 7 contained within the first interior space 5 of each of the at least one additional confinement structure 3, wherein the material 7 is a synthetic polymer capable of selectively binding the first or further analyte, a reference material, or an electrolyte. FIG. 8 shows an example of such a sensor 1.

FIG. 8 shows a sensor having four confinement structures 3a-3d on a substrate 2 both (a) before and (b) after the incorporation of the synthetic polymer capable of selectively binding the first or further analyte, a reference material, or an electrolyte. Each of the confinement structures 3a-3d contains a transducer 6a-6d. The first confinement structure 3a is fabricated from a first limiting structure 4a and a second limiting structure 8a, and contains a transducer 6a. The second confinement structure 3b has only a first limiting structure 4b which is fabricated at the same time as the second limiting structure Sa of the first confinement structure 3a. The break in the drawing of the sensor indicates that any number of additional confinement structures may be incorporated. Confinement structures 3c and 3d are analogous to confinement structures 3b and 3a, respectively.

With reference to FIG. 8(b), the first confinement structure 3a includes, for example, a MIP 7a capable of selectively binding a first analyte and a cover layer 11a. The second confinement structure 3b includes, for example, a MIP 7b capable of selectively binding a second analyte only. The third confinement structure 3c may include a non-imprinted polymer otherwise identical in composition to MIP 7b in order to make a reference measurement. The fourth confinement structure 3d may include a MIP 7d capable of selectively binding a third analyte and a cover layer 11d.

The reference measurement which may be taken in confinement structure 3c is an additional advantage of the sensor of the present invention. The measurement may be obtained by carrying out a differential measurement on two transducers 6b and 6c one of which is coated with a MIP 7b and the other is coated with a material 7c of identical composition, polymerised and/or cross-linked in the absence of the template molecule, i.e. a corresponding non-imprinted polymer. The reason for this is that a MIP may have, besides the binding sites specific to the analyte(s) to be detected, non-specific sites which can bind other molecules. On the other hand, the material polymerised in the absence of the template possesses only non-specific sites. It is thus possible to compensate either fully or partially for the interference which may be due to molecules other than the analyte(s), which become bound to the MIP 7b by non-specific interactions. This would typically be a material made from the same composition as the MIP, but not imprinted, i.e. polymerised in the absence of the template.

In the simplest embodiment, the reference signal may be subtracted from that of the signal from the functionalised polymer, e.g. MI[P. However, more elaborate compensation schemes are known in the art.

The sensor 1 of the present invention allows the measurement, either separately or simultaneously, of related metabolites of the analyte(s) of interest to give information on the physiological passage/pharmacokinetics of the analyte(s). For example, in the case of propofol, one could detect metabolites or derivates of metabolites of propofol, such as propofol-glucuronide, 2,6-diisopropyl-1,4-quinol, 4-(2,6-diisopropyl-1,4-quinol)-sulphate, 1- or 4-(2,6-diisopropyl-1,4-quinol)-glucuronide.

A further embodiment of the present invention employs a differential measurement at two transducers with the same polymers, one of which uses a partitioning material, while the second is fabricated without the partitioning material.

The confinement structures of the present invention may also be included in sensors with have other biosensors and chemical sensors known in the art, such as those described in U.S. Pat. No. 5,376,255 and U.S. Pat. No. 5,376,2565.

The analyte which is detected by the synthetic polymer, exemplified by a MIP, is usually the same as the target species which is the subject of the analysis. For example, in the analysis of the target species propofol, propofol itself is also the analyte which interacts with the synthetic polymer which has previously been synthesised to bind selectively to propofol. However, the sensor of the present invention may also be used to detect an analyte which is not the same of the target species of interest. The analyte may be, for example, a metabolite or other derivative of the target species of interest.

The sensor may also be used as a competitive assay. In this mode of operation, the synthetic polymer responds to a target species which displaces the analyte from the synthetic polymer. In this case, the signal received by the detector will decrease in the presence of the target species of interest as the analyte is displaced from the synthetic polymer. In one particular embodiment, the target species of interest may react with the analyte. The sensor may also be used as a sandwich assay, i.e. the analyte binds to the synthetic polymer and then a label binds to the analyte or analyte/polymer complex.

The sensor of the present invention is typically incorporated into a sampling system and a signal processing unit. An example of such a system is shown in FIG. 9. The system is equipped with a housing 12 incorporating the sensor 1 coupled to a sampling port 13 in an intravascular line 14 above the sensor 1. A sampling device 15, for example a syringe, is coupled to the sampling port 13. Using the sampling device 15, the user will withdraw blood flushing it across the sensor 1 in order to take a measurement. After the measurement is completed, the blood may be flushed back into the patient or it may be flushed to waste. In another embodiment, the sensor can be incorporated into the intravascular-flushing line, for example, along with one or more other sensors, such as a pressure sensor. Samples may be taken either periodically, regularly, event-driven, on demand or following a user intervention.

The sensor 1 is connected to a local display and signal processing unit 16 which may be connected to a patient monitoring device 17. The sensor I is also connected to the housing 12 electronically using techniques known in the art.

In addition to the system described above, the sensor may be employed in a range of other sensing systems, known to those skilled in the art. For example, rather than being directly connected to the patient, a sample may be taken from the patient and transported to and injected into an analyser, into which the sensor is integrated, for sample analysis.

In addition to providing detection and measurements of markers, substances or drugs, the sensor of the present invention provides feedback for the treatment of the patient based on the results of the analysis made. This feedback may be provided either directly to the user or it may be part of a closed-loop control system including the device administering the treatment to the patient. One particular example is a sensor for an anaesthetic agent, such as propofol, which measures the concentration of the anaesthetic agent in one or more bodily fluids or body compartments, e.g. blood or blood plasma, and based on these measurements directs, either directly or the user, the subsequent delivery of the anaesthetic agent, e.g. by controlling the rate of delivery to the patient via a syringe pump.

The sensor may also be used with systems which monitor other parameters which characterise the health of a patient, monitor particular markers indicating disease states or direct the patient's treatment, e.g. blood gases, pH, temperature etc.

An embodiment of the present invention relates to a propofol sensor. In this case, a propofol-imprinted MIP was prepared by polymerising a solution of methacrylic acid (MAA) using ethylenedimethylacrylic acid (EDMA) as a cross-linker and 1,1′-azobis (cyclohexanecarbonitrile) (ABCHC) as the initiator. All chemicals were dissolved in hexane with the ratio of propofol:MAA:EDMA:ABCHC of 1:4:30:0.17 (at 1.65 ml/g hexane). A droplet of the solution was deposited into a double-limiting structure formed around an amperometric transducer on the substrate and thermally polymerised at 60-80° C. for 24 h. After polymerisation, the chip surface was washed with methanol and hexane to remove the propofol template from the polymer.

A droplet of a solution consisting of 2.374 g of hydroxymethyl methacrylate resin (HEMA), 0.025 g of dimethoxyphenylacetophenone (DMAP) and 0.075 g of tetraethylene glycol methacrylate (TEGM) in 60 μl of triethylene glycol was then deposited into the second interior space and on top of the propofol-imprinted MIP. This material was then polymerised using UV-light to form a membrane covering the propofol-imprinted MIP.

The sensor was brought into contact with an aqueous saline solution of 6 μM propofol. The propofol was bound by the polymer layer on top of the amperometric transducer and was detected using voltammetry, see FIG. 10.

Although the majority of routine clinical measurements are taken from blood, other bodily fluids may be sampled. In addition, the sensor 1 of the present invention may also be used to detect the presence of analytes in exhaled breath. Measurement of breath-borne analytes has the advantage that breath concentration is closely related to the blood concentration at the alveoli and has the potential to measure rapid changes in real time. The sensor of the present invention may therefore also be placed into the endotracheal tube, anaesthetic circuit or ventilator circuit of a patient. In such a system, it may be advantageous to agitate and/or oscillate the exhaled air in the artificial airway to aid diffusion of the analytes of interest from the alveolus and facilitate greater concentration of the analyte at the sensor. The sensor may also employ a cover layer to concentrate the analyte in the exhaled breath.

In a further embodiment, the sensor of the present invention may be used for the detection of ischaemia modified albumin (IMA). Acute myocardial infarction (AMI) is a condition caused by blockage of the blood vessels supplying the heart muscle. The resulting lack of oxygen can cause permanent damage to the heart muscle (infarction) leading to illness and in many cases death. The effects of AMI can be limited or prevented by rapid treatment.

While many techniques exist to determine whether a patient has suffered from AMI, these tend to have a low degree of specificity leading to false negative diagnoses and are slow permitting further damage to the heart muscle.

An emerging marker for the early detection of AMI is IMA. Human serum albumin (albumin) is a significant component of whole blood (˜40 g per litre) and plays an important role in the transport of lipids and the regulation of osmotic pressure. It has been observed that a specific binding site on the albumin is altered by ischaemia, modifying the ability for albumin to bind metal ions at this site. This has lead to the Albumin Cobalt Binding (ACB®) test being developed to measure the concentration of IMA in blood for early detection of an ischaemic event, and there is a growing body of evidence that this is an effective test for the rapid detection of AMI.

The ACB® test involves determining the overall albumin concentration in a blood sample, adding a sufficient amount of cobalt ions to bind to the albumin and then calorimetrically determining the quantity of unbound cobalt to infer a concentration of IMA. Variants have been described to this approach, including use of other metal ions or fluorescent markers that bind to the inmodified albumin, and the measurement is made in an equivalent way. All require substantial sample preparation and are as such a test to be carried out in an analytical laboratory environment.

It is evident that a method either to directly measure the IMA, or to make the two measurements by which the IMA concentration is inferred in a differential manner is preferable for a number of reasons. Firstly, direct measurement of the specific target inherently generates less error than measuring the difference between two large concentrations (albumin concentration, cobalt concentration). Secondly, such methods can more easily be configured into a point-of-care (PoC) instrument to be used by front line clinical staff rather than skilled laboratory personnel. Speed of results is critical in the successful treatment of patients and a PoC instrument removes the time associated with transferring a sample to the laboratory and the results back to the clinical staff. Thirdly, such methods may be compatible with use for repeated measurements in an arterial, venous or other line (e.g. a drain) attached to the patient. Patients are likely to have such an access and generally require a number of measurements to be made over hours or days. An on-line on-demand system has a number of benefits including very rapid turnaround time for tests, increased frequency of measurement which may additionally be used to provide a trend analysis, lowering of infection risk to both patient and care giver and conservation of blood volume.

The sensor of the present invention allows the measurement of IMA concentration in a patient's biological sample, such as blood, plasma, serum, saliva, interstitial fluid, dialysate or other fluid, which may optionally be purified to remove, for example, red blood cells, platelets etc. The sensor may be integrated into a patient connected device, as described hereinabove.

In a preferred embodiment the method uses a sensor to measure IMA directly. The sensor includes an IMA-sensitive synthetic polymer, such as an MIP.

In another embodiment, a sample of the fluid to be analysed is mixed with a known quantity of transition metal ions in excess of the total concentration of albumin in the sample. The metal ion is preferably selected from Groups 1b-7b or 8 of the periodic table, including the group V, As, Co, Sb, Cr, Mo, Mn, Ba, Zn, Ni, Hg, Cd, Fe, Pb, Au and Ag. The sample is left for sufficient time for unmodified albumin to react with the metal ions. A sensor is used to determine the residual concentration of unbound metal ions. The unbound metal ions bind to, or in some other way interact with, the synthetic polymer in the sensor. The synthetic polymer may, for example, be a MIP or a porphyrin ionophore contained within a PVC matrix to detect Co²⁺ ions.

Possible combinations within the sensor include the detection of IMA and total albumin, IMA and for one or more interfering species, total albumin and residual metal ions, or combinations thereof. The sensor may have capability for just for IMA, or preferably a panel of analytes including, for example, cardiac troponin markers.

1. A sensor comprising:
a substrate;
a confinement structure created from materials applied to the substrate by deposition, wherein the confinement structure comprises at least a first limiting structure defining a first interior space;
a transducer proximal to the first interior space; and
a first synthetic polymer capable of selectively binding a first analyte, within the confinement structure.

2. A sensor as claimed in claim 1, wherein the confinement structure further comprises a second limiting structure defining a second interior space, the second interior space containing the first interior space.

3. A sensor as claimed in claim 2, wherein the confinement structure further comprises one or more further limiting structures defining one or more further interior spaces, the one or more further interior spaces each containing an interior space.

4. A sensor as claimed in claim 1, wherein the first synthetic polymer capable of selectively binding a first analyte is disposed in the first interior space.

5. A sensor as claimed in claim 3, wherein the first synthetic polymer capable of selectively binding a first analyte is disposed in a space selected from the group consisting of: the second interior space, and a further interior space.

6. A sensor as claimed in claim 1, wherein the internal diameter of the first limiting structure is about 10-350 μm.

7. A sensor as claimed in claim 1, wherein height of the first limiting structure is about 1-10 μm.

8. A sensor as claimed in claim 2, wherein the internal diameter of the second limiting structure is about 50-600 μm.

9. A sensor as claimed in claim 2, wherein the height of the second limiting structure is about 1-100 μm.

10. A sensor as claimed in claim 2, wherein the limiting structures of the confinement structure are annular.

11. A sensor as claimed in claim 1, wherein the sensor further comprises:
at least one additional confinement structure;
a transducer proximal to the first interior space of each of the at least one additional confinement structures; and
a material contained within the at least one additional confinement structure, wherein the material is selected from the group consisting of: the synthetic polymer capable of selectively binding a first analyte, a further synthetic polymer capable of selectively binding a further analyte, and a reference material.

12. A sensor as claimed in claim 1, wherein the first synthetic polymer is a molecularly imprinted polymer.

13. A sensor as claimed in claim 1, wherein the first synthetic polymer is a polymer capable of selectively binding a substance selected from the group consisting of: morphine, propofol, an antibiotic, and IMA.

14. A sensor as claimed in claim 11, wherein the further synthetic polymer is a molecularly imprinted polymer.

15. A sensor as claimed in claim 11, wherein the sensor comprises at least one additional confinement structure having a reference material therein, and the first synthetic polymer is a molecularly imprinted polymer and the reference material is a corresponding non-imprinted polymer.

16. A sensor as claimed in claim 1 wherein a space selected from the first, second and a further interior space contains a material selected from the group consisting of a conducting material and a mediator.

17. A sensor as claimed in claim 16, wherein the conducting material is an electrolyte.

18. A sensor as claimed in claim 1, wherein the at least one confinement structure further comprises one or more additional substances which provide a specific environment therein.

19. A sensor as claimed in claim 18, wherein the specific environment is a non-aqueous environment.

20. A sensor as claimed in claim 1, wherein the transducer is disposed on the substrate.

21. A sensor as claimed in claim 1, wherein the transducer is selected from the group consisting of: an electrochemical, conductimetric, optical, fluorescent, luminescent, absorption, time-of-flight, gravimetric, strain or displacement, surface-acoustic wave, resonant, thermal transducer, and combinations thereof.

22. A sensor as claimed in claim 1, wherein the substrate is a silicon wafer.

23. A sensor as claimed in claim 1, wherein the substrate is substantially planar.

24. A sensor as claimed in claim 1 wherein the confinement structure is fabricated from a polyimide.

25. A method of detecting a target species in a sample comprising contacting a sensor as claimed in claim 1 with a sample containing or suspected to contain the target species.

26. A method as claimed in claim 25, wherein the sample is returned to the patient.

27. A method as claimed in claim 25, wherein the sample is not returned to the
